(19) United States
(12) Patent Application Publication
(10) Pub. No.: US 2015/0171338 A1
(43) Pub. Date: Jun. 18, 2015

(54) MATERIAL FOR ORGANIC ELECTROLUMINESCENCE DEVICE AND ORGANIC ELECTROLUMINESCENCE DEVICE INCLUDING THE SAME

(71) Applicant: SAMSUNG DISPLAY CO., LTD., Yongin-City (KR)

(72) Inventors: Naoya SAKAMOTO, Yokohama (JP); Yasuo MIYATA, Yokohama (JP)

(21) Appl. No.: 14/573,610

(22) Filed: Dec. 17, 2014

(30) Foreign Application Priority Data
Dec. 18, 2013 (JP) 2013-261647

(51) Int. Cl. H01L 51/00; C07D 491/048; H01L 51/50; C07D 495/04

(52) U.S. Cl. CPC H01L 51/0061; C07D 495/04; H01L 51/0071; H01L 51/0072; C07D 491/048; H01L 51/5012

(57) ABSTRACT

A compound for an organic electroluminescence device and an organic electroluminescence device, the compound including a triarylamine moiety, and a heterocyclic moiety represented by the following Formula (1):

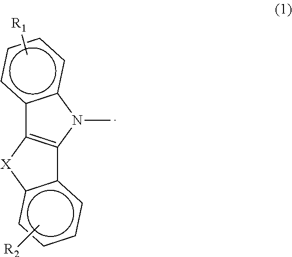

### CROSS-REFERENCE TO RELATED APPLICATION

Japanese Patent Application No. 2013-261647, filed on Dec. 18, 2013, in the Japanese Patent Office, and entitled: “Material for Organic Electroluminescence Device and Organic Electroluminescence Device Including the Same,” is incorporated by reference herein in its entirety.

### BACKGROUND

1. Field

Embodiments relate to a material for an organic electroluminescence device and an organic electroluminescence device including the same.

2. Description of the Related Art

As an image display apparatus, an organic electroluminescence display (organic EL display) is being actively developed.

The organic EL display is, unlike a liquid crystal display or the like, a self-emitting type display that embodies displaying through light emission of a light emitting material including an organic compound of the light emitting layer by recombining holes and electrons injected from an anode and a cathode in a light emitting layer.

An example of an organic electroluminescence device (organic EL device) may include an anode, a hole transport layer disposed on the anode, a light emitting layer disposed on the hole transport layer, an electron transport layer disposed on the light emitting layer, and a cathode disposed on the electron transport layer. Holes may be injected from the anode, and the injected holes may be injected into the light emitting layer through the hole transport layer. Meanwhile, electrons may be injected from the cathode, and the injected electrons may be injected into the light emitting layer through the electron transport layer. The holes and the electrons injected into the light emitting layer may be recombined, and an exciton may be generated in the light emitting layer. The organic electroluminescence device may emit light by using light generated while the exciton returns to a ground state.

### SUMMARY

Embodiments are directed to a material for an organic electroluminescence device and an organic electroluminescence device including the same.

The embodiments may be realized by providing a compound for an organic electroluminescence device, the compound including a triarylamine moiety, and a heterocyclic moiety represented by the following Formula (1):

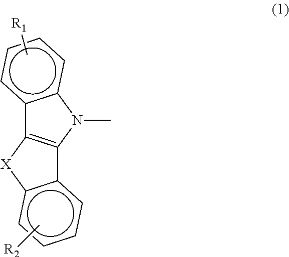

wherein, in Formula (1), X is an oxygen atom or a sulfur atom, and R₁ and R₂ are each independently a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom

The embodiments may be realized by providing an organic electroluminescence device including an anode; a cathode; a light emitting layer between the anode and the cathode; and at least one stacking layer between the anode and the light emitting layer, wherein the at least one stacking layer includes the compound for an organic electroluminescence device according to an embodiment.

The embodiments may be realized by providing an organic electroluminescence device including an anode; a cathode; a light emitting layer between the anode and the cathode; and at least one stacking layer between the anode and the light emitting layer, wherein the light emitting layer includes the compound for an organic electroluminescence device according to an embodiment.

The embodiments may be realized by providing a compound for an organic electroluminescence device, the compound being represented by the following Formula (2):

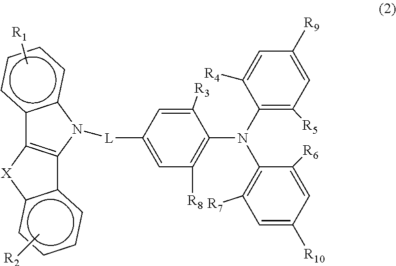

wherein, in Formula (2), X is an oxygen atom or a sulfur atom, R₁ and R₂ are each independently a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom, R₃ to R₁₀ are each independently a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom, and L is a divalent arylene group.

At least two adjacent ones of R₃ to R₈ may be connected to each other to form a saturated or unsaturated ring.

The embodiments may be realized by providing an organic electroluminescence device including an anode; a cathode; a light emitting layer between the anode and the cathode; and at least one stacking layer between the anode and the light emitting layer, wherein the at least one stacking layer includes the compound for an organic electroluminescence device according to an embodiment.

The embodiments may be realized by providing an organic electroluminescence device including an anode; a cathode; a light emitting layer between the anode and the cathode; and at least one stacking layer between the anode and the light emitting layer, wherein the light emitting layer includes the compound for an organic electroluminescence device according to an embodiment.

### BRIEF DESCRIPTION OF THE DRAWING

Features will be apparent to those of skill in the art by describing in detail exemplary embodiments with reference to the attached drawing in which:

FIG. 1 illustrates a diagram of an organic electroluminescence device according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

Lower driving voltage or higher efficiency may be expected through combining an indolobenzopyrrole moiety and a triarylamine moiety. A further reduction in a HOMO (highest occupied molecular orbital) level may be achieved when a compound including the indolobenzopyrrole moiety and the triarylamine moiety are included in a stacking layer between a light emitting layer and an anode and/or in the light emitting layer. In addition, significantly lower driving voltage and/or higher efficiency may be achieved by introducing an indolobenzofuran moiety or an indolobenzothiophene moiety, instead of the indolobenzopyrrole moiety, into or onto a triarylamine moiety.

The compound for an organic electroluminescence device according to an embodiment may include a triarylamine moiety and a heterocyclic moiety represented by the following Formula (1).

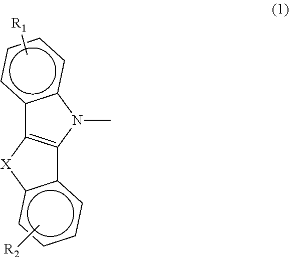

In Formula (1), X may be an oxygen atom or a sulfur atom, and R₁ and R₂ may each independently be or include a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom.

In an implementation, R₁ and R₂ may each independently be or include, e.g., any one of a methyl group, an ethyl group, an n-propyl group, an i-propyl group, an n-butyl group, a sec-butyl group, a tert-butyl group, a phenyl group, a tolyl group, and a biphenyl group.

The compound for an organic electroluminescence device according to an embodiment may have the indolobenzofuran moiety or the indolobenzothiophene moiety in or on the triarylamine moiety, and may be used to form an organic electroluminescence device driven at a low voltage with high efficiency. The compound for an organic electroluminescence device according to the embodiment may help reduce the HOMO level, as compared to other materials, and may be suitable for use as a material of a stacking layer disposed between the light emitting layer and the anode, e.g., a hole transport material, by including the indolobenzofuran moiety or indolobenzothiophenyl moiety, instead of an indolobenzopyrrole moiety. In an implementation, the triarylamine moiety may include a carbazole moiety, e.g., may include a carbazole moiety with an N-bound aryl group.

In an implementation, the compound for an organic electroluminescence device may be represented by the following Formula (2).

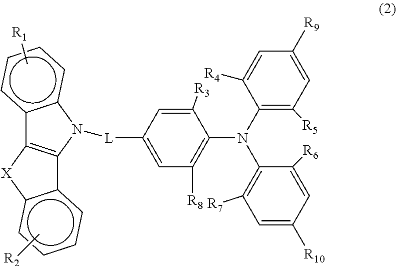

In Formula (2), X may be, e.g., an oxygen atom or a sulfur atom, and R₁ and R₂ may each independently be or include, e.g., a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom. In an implementation, R₁ and R₂ may include the examples described above with respect to Formula (1).

In Formula (2), R₃ to R₁₀ may each independently be or include, e.g., a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom. In an implementation, R₃ to R₁₀ may be different from each other, or two or more substituent groups may be the same.

In Formula (2), L may be a divalent arylene group. In an implementation, L may be, e.g., an arylene group having 6 to 18 ring carbon atoms.

In an implementation, in Formula (2), adjacent ones of R₃ to R₈ may be connected to each other, and may form a saturated or unsaturated cycle or ring. In an implementation, in Formula (2), adjacent ones of R₃ to R₈ may be an electron pair that forms a single bond between ring carbons of the phenyl rings. For example, the ring carbon of R₃ may be single bonded to the ring carbon of R₄, e.g., forming a carbazole moiety. R₃ to R₈ may be connected to each other and form the saturated or unsaturated cycle to form the organic electroluminescence device driven at a low voltage with high efficiency.

The compound for an organic electroluminescence device according to an embodiment may include an indolobenzofuran group, e.g., may be represented by the following Formula (5), or may include an indolobenzothiophene group, e.g, may be represented by Formula (6).

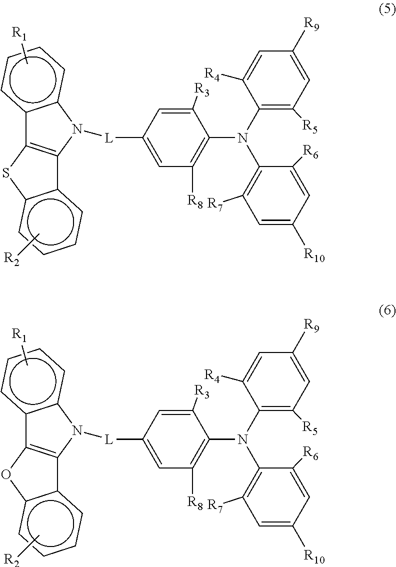

In an implementation, R₃ to R₁₀ may each independently be or include, e.g., any one of a methyl group, an ethyl group, a n-propyl group, an i-propyl group, a n-butyl group, a sec-butyl group, a tert-butyl group, a phenyl group, a tolyl group, a dimethylphenyl group, a trimethylphenyl group, a diphenylphenyl group, a triphenylphenyl group, a fluorophenyl group, a difluorophenyl group, a trifluorophenyl group, a tetrafluorophenyl group, a pentafluorophenyl group, a biphenyl group, a naphthyl group, a naphthylphenyl group, a phenylnaphthyl group, a pyridyl group, a pyridylphenyl group, a quinolyl group, a trimethylsilylphenyl group, and a triphenylsilylphenyl group.

L may be a divalent arylene group, e.g., any one of a phenylene group, biphenylene, a fluorenylene group, a naphthylene group, and an anthracene group.

In an implementation, the compound for an organic electroluminescence device according to an embodiment may be any one of the following Compounds 1 to 68.

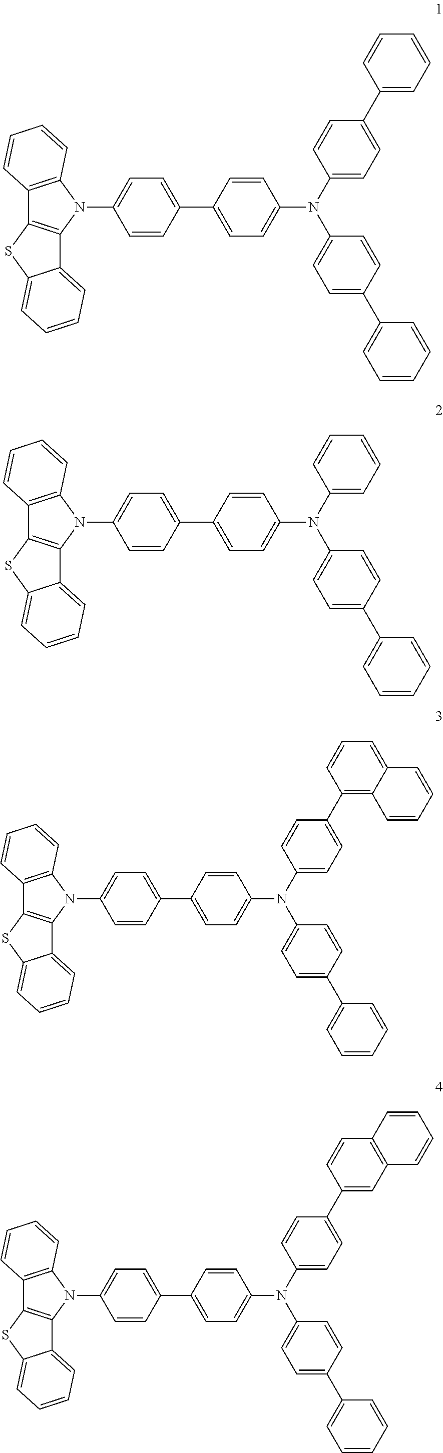 [SMILES: c1ccc(-c2ccc(N(c3ccc(-c4ccccc4)cc3)c3ccc(-c4ccc(-n5c6ccccc6c6sc7ccccc7c65)cc4)cc3)cc2)cc1 ; c1ccc(-c2ccc(N(c3ccccc3)c3ccc(-c4ccc(-n5c6ccccc6c6sc7ccccc7c65)cc4)cc3)cc2)cc1 ; c1ccc(-c2ccc(N(c3ccc(-c4ccc(-n5c6ccccc6c6sc7ccccc7c65)cc4)cc3)c3ccc(-c4cccc5ccccc45)cc3)cc2)cc1 ; c1ccc(-c2ccc(N(c3ccc(-c4ccc(-n5c6ccccc6c6sc7ccccc7c65)cc4)cc3)c3ccc(-c4ccc5ccccc5c4)cc3)cc2)cc1]

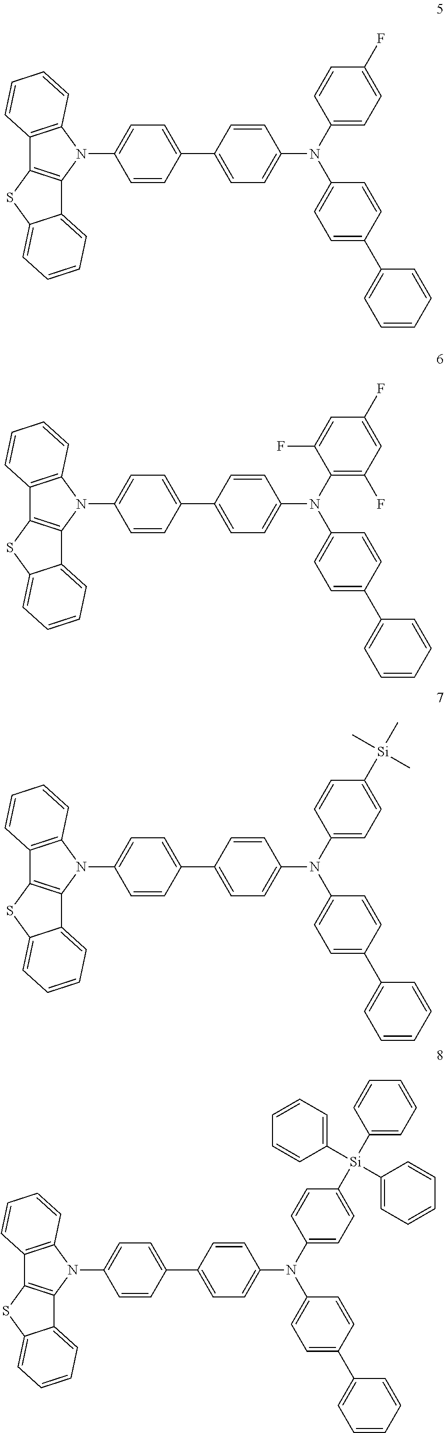

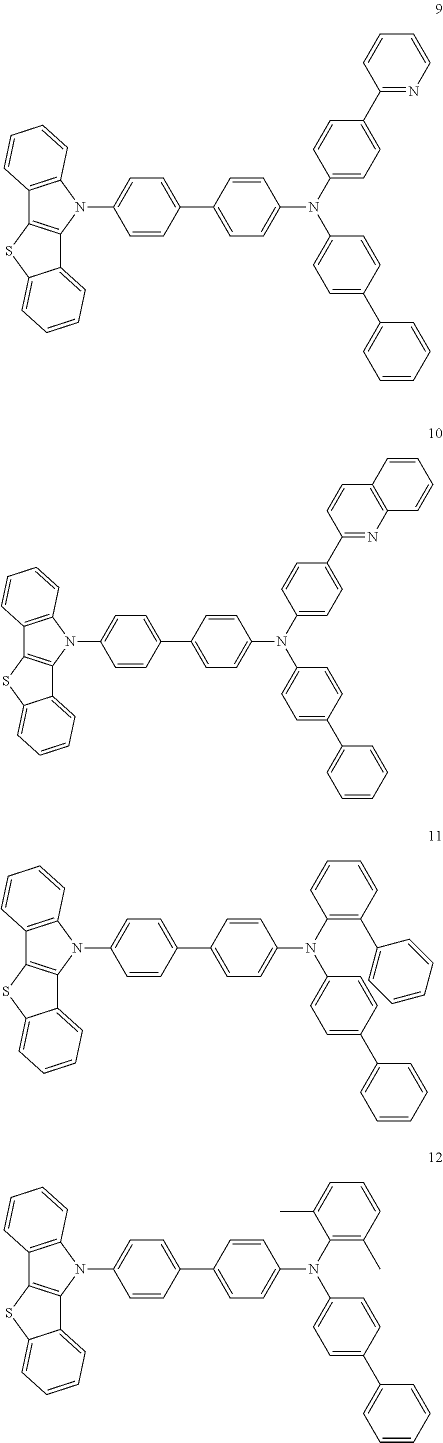 [SMILES: c1ccc(-c2ccc(N(c3ccc(-c4ccc(-n5c6ccccc6c6sc7ccccc7c65)cc4)cc3)c3ccc(-c4ccccn4)cc3)cc2)cc1 ; c1ccc(-c2ccc(N(c3ccc(-c4ccc(-n5c6ccccc6c6sc7ccccc7c65)cc4)cc3)c3ccc(-c4ccc5ccccc5n4)cc3)cc2)cc1 ; c1ccc(-c2ccc(N(c3ccc(-c4ccc(-n5c6ccccc6c6sc7ccccc7c65)cc4)cc3)c3ccccc3-c3ccccc3)cc2)cc1 ; Cc1cccc(C)c1N(c1ccc(-c2ccccc2)cc1)c1ccc(-c2ccc(-n3c4ccccc4c4sc5ccccc5c43)cc2)cc1]

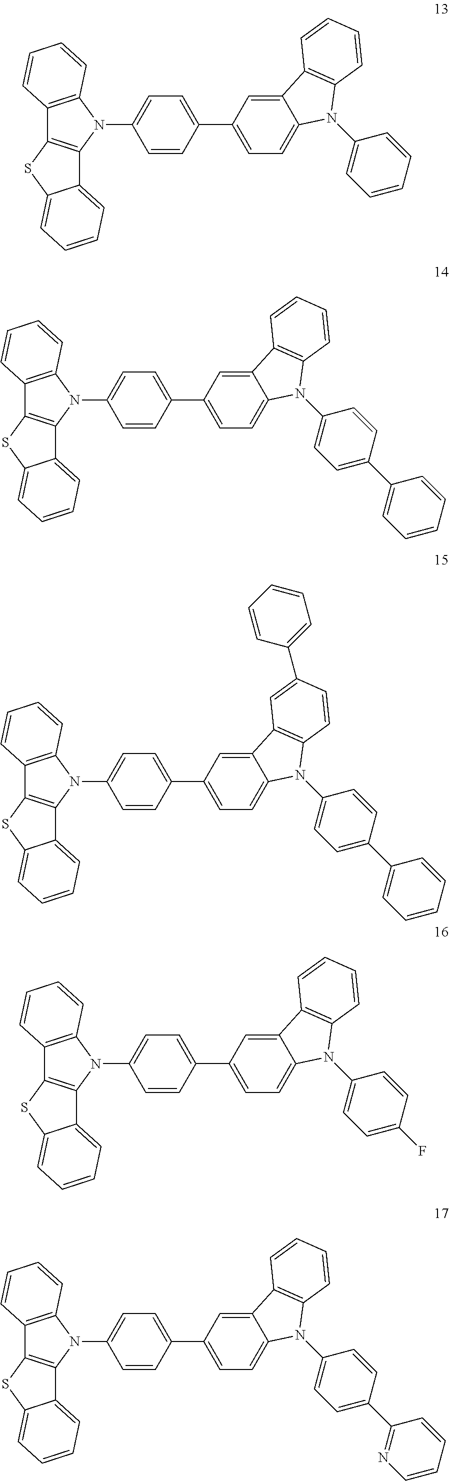 [SMILES: c1ccc(-n2c3ccccc3c3cc(-c4ccc(-n5c6ccccc6c6sc7ccccc7c65)cc4)ccc32)cc1 ; c1ccc(-c2ccc(-n3c4ccccc4c4cc(-c5ccc(-n6c7ccccc7c7sc8ccccc8c76)cc5)ccc43)cc2)cc1 ; c1ccc(-c2ccc(-n3c4ccc(-c5ccccc5)cc4c4cc(-c5ccc(-n6c7ccccc7c7sc8ccccc8c76)cc5)ccc43)cc2)cc1 ; Fc1ccc(-n2c3ccccc3c3cc(-c4ccc(-n5c6ccccc6c6sc7ccccc7c65)cc4)ccc32)cc1 ; c1ccc(-c2ccc(-n3c4ccccc4c4cc(-c5ccc(-n6c7ccccc7c7sc8ccccc8c76)cc5)ccc43)cc2)nc1]

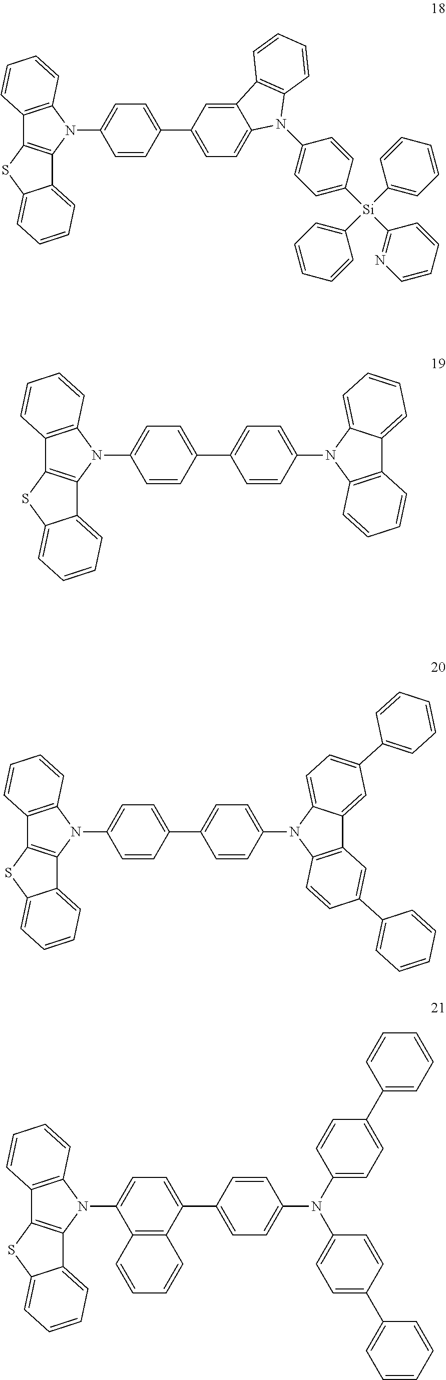 [SMILES: c1ccc([Si](c2ccccc2)(c2ccc(-n3c4ccccc4c4cc(-c5ccc(-n6c7ccccc7c7sc8ccccc8c76)cc5)ccc43)cc2)c2ccccn2)cc1 ; c1ccc2c(c1)sc1c3ccccc3n(-c3ccc(-c4ccc(-n5c6ccccc6c6ccccc65)cc4)cc3)c21 ; c1ccc(-c2ccc3c(c2)c2cc(-c4ccccc4)ccc2n3-c2ccc(-c3ccc(-n4c5ccccc5c5sc6ccccc6c54)cc3)cc2)cc1 ; c1ccc(-c2ccc(N(c3ccc(-c4ccccc4)cc3)c3ccc(-c4ccc(-n5c6ccccc6c6sc7ccccc7c65)c5ccccc45)cc3)cc2)cc1]

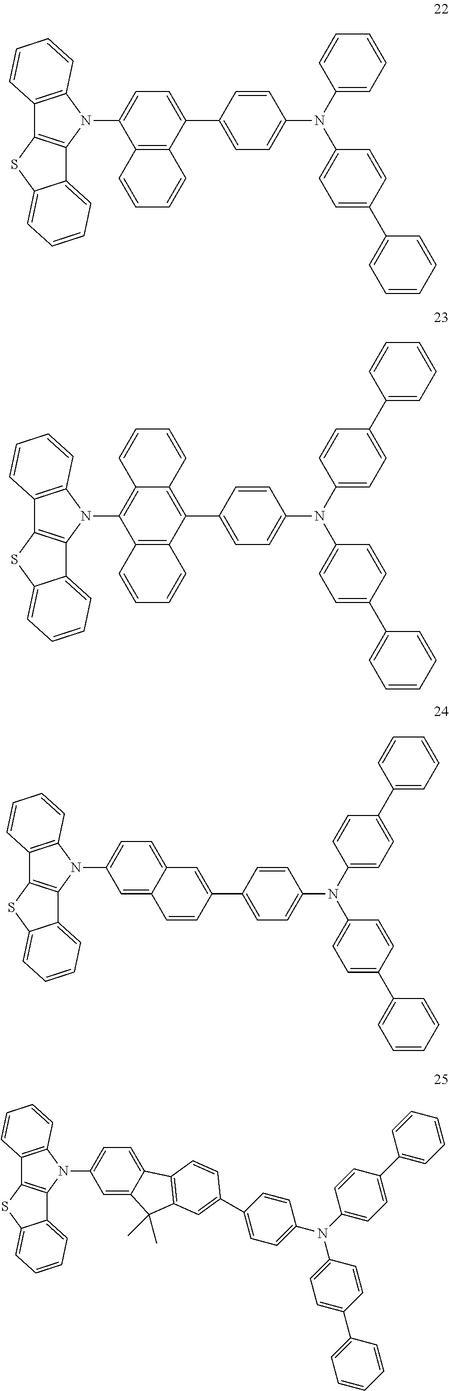 [SMILES: c1ccc(-c2ccc(N(c3ccccc3)c3ccc(-c4ccc(-n5c6ccccc6c6sc7ccccc7c65)c5ccccc45)cc3)cc2)cc1 ; c1ccc(-c2ccc(N(c3ccc(-c4ccccc4)cc3)c3ccc(-c4c5ccccc5c(-n5c6ccccc6c6sc7ccccc7c65)c5ccccc45)cc3)cc2)cc1 ; c1ccc(-c2ccc(N(c3ccc(-c4ccccc4)cc3)c3ccc(-c4ccc5cc(-n6c7ccccc7c7sc8ccccc8c76)ccc5c4)cc3)cc2)cc1 ; CC1(C)c2cc(-c3ccc(N(c4ccc(-c5ccccc5)cc4)c4ccc(-c5ccccc5)cc4)cc3)ccc2-c2ccc(-n3c4ccccc4c4sc5ccccc5c43)cc21]

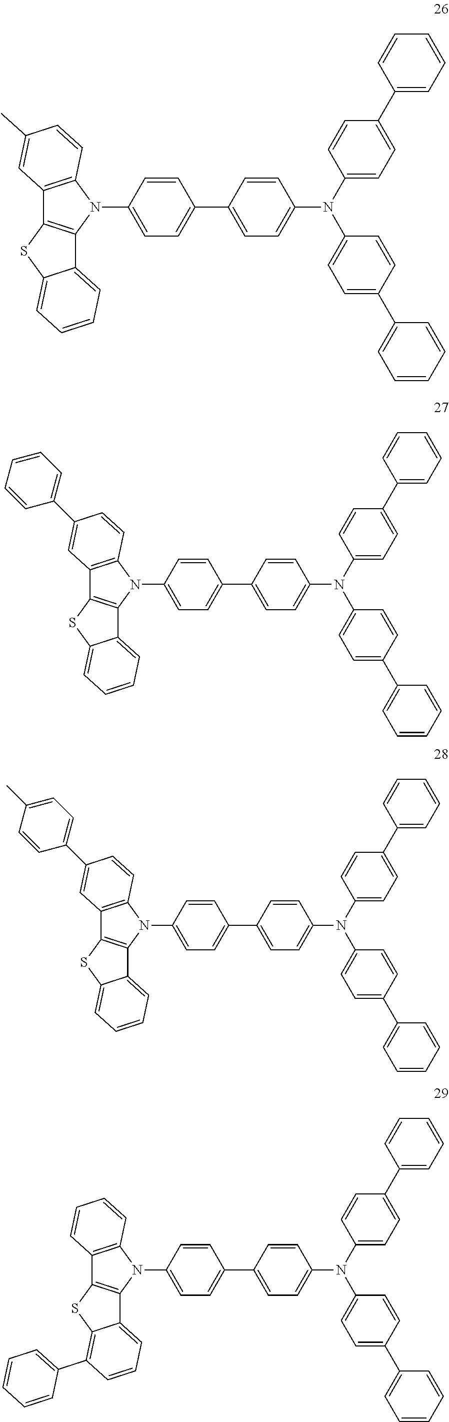 [SMILES: Cc1ccc2c(c1)c1sc3ccccc3c1n2-c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4ccc(-n5c6ccc(-c7ccccc7)cc6c6sc7ccccc7c65)cc4)cc3)cc2)cc1 ; Cc1ccc(-c2ccc3c(c2)c2sc4ccccc4c2n3-c2ccc(-c3ccc(N(c4ccc(-c5ccccc5)cc4)c4ccc(-c5ccccc5)cc4)cc3)cc2)cc1 ; c1ccc(-c2ccc(N(c3ccc(-c4ccccc4)cc3)c3ccc(-c4ccc(-n5c6ccccc6c6sc7c(-c8ccccc8)cccc7c65)cc4)cc3)cc2)cc1]

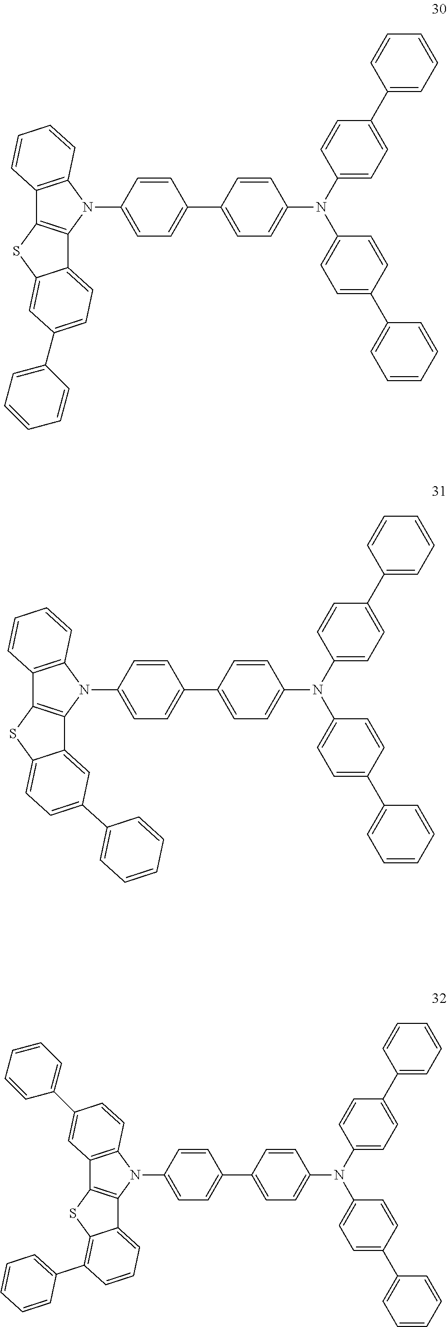 [SMILES: c1ccc(-c2ccc(N(c3ccc(-c4ccccc4)cc3)c3ccc(-c4ccc(-n5c6ccccc6c6sc7cc(-c8ccccc8)ccc7c65)cc4)cc3)cc2)cc1 ; c1ccc(-c2ccc(N(c3ccc(-c4ccccc4)cc3)c3ccc(-c4ccc(-n5c6ccccc6c6sc7ccc(-c8ccccc8)cc7c65)cc4)cc3)cc2)cc1 ; c1ccc(-c2ccc(N(c3ccc(-c4ccccc4)cc3)c3ccc(-c4ccc(-n5c6ccc(-c7ccccc7)cc6c6sc7c(-c8ccccc8)cccc7c65)cc4)cc3)cc2)cc1]

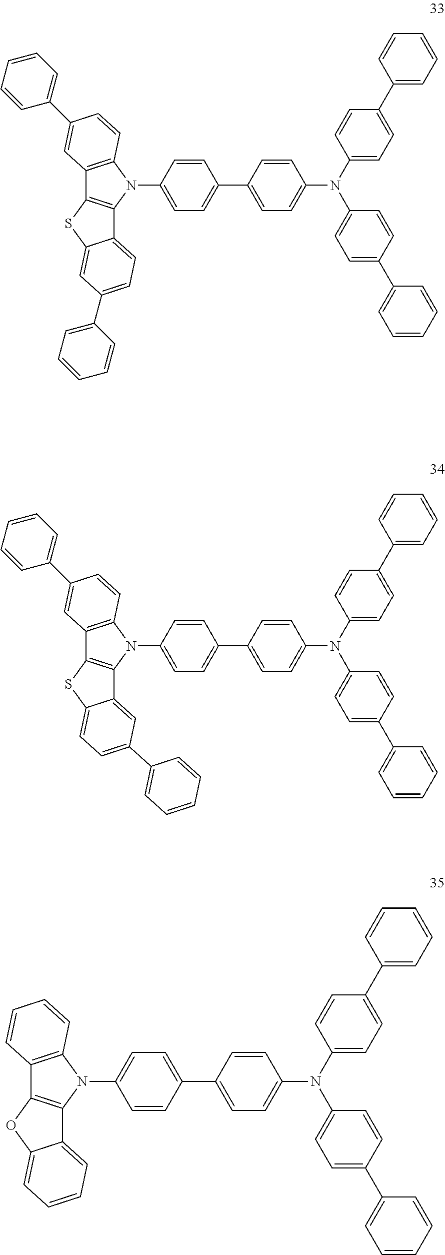 [SMILES: c1ccc(-c2ccc(N(c3ccc(-c4ccccc4)cc3)c3ccc(-c4ccc(-n5c6ccc(-c7ccccc7)cc6c6sc7cc(-c8ccccc8)ccc7c65)cc4)cc3)cc2)cc1 ; c1ccc(-c2ccc(N(c3ccc(-c4ccccc4)cc3)c3ccc(-c4ccc(-n5c6ccc(-c7ccccc7)cc6c6sc7ccc(-c8ccccc8)cc7c65)cc4)cc3)cc2)cc1 ; c1ccc(-c2ccc(N(c3ccc(-c4ccccc4)cc3)c3ccc(-c4ccc(-n5c6ccccc6c6oc7ccccc7c65)cc4)cc3)cc2)cc1]

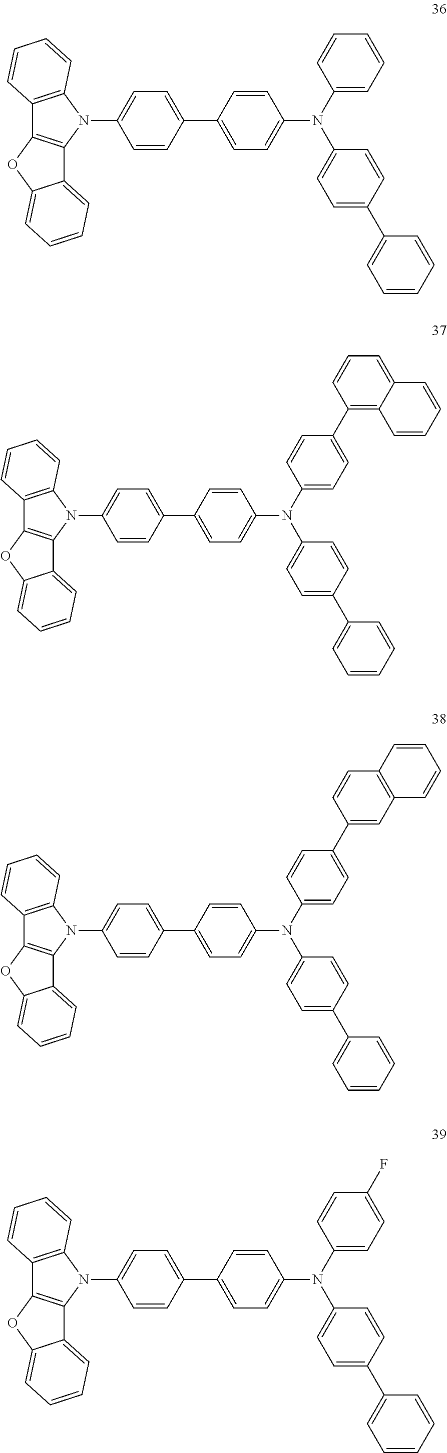 [SMILES: c1ccc(-c2ccc(N(c3ccccc3)c3ccc(-c4ccc(-n5c6ccccc6c6oc7ccccc7c65)cc4)cc3)cc2)cc1 ; c1ccc(-c2ccc(N(c3ccc(-c4ccc(-n5c6ccccc6c6oc7ccccc7c65)cc4)cc3)c3ccc(-c4cccc5ccccc45)cc3)cc2)cc1 ; c1ccc(-c2ccc(N(c3ccc(-c4ccc(-n5c6ccccc6c6oc7ccccc7c65)cc4)cc3)c3ccc(-c4ccc5ccccc5c4)cc3)cc2)cc1 ; Fc1ccc(N(c2ccc(-c3ccccc3)cc2)c2ccc(-c3ccc(-n4c5ccccc5c5oc6ccccc6c54)cc3)cc2)cc1]

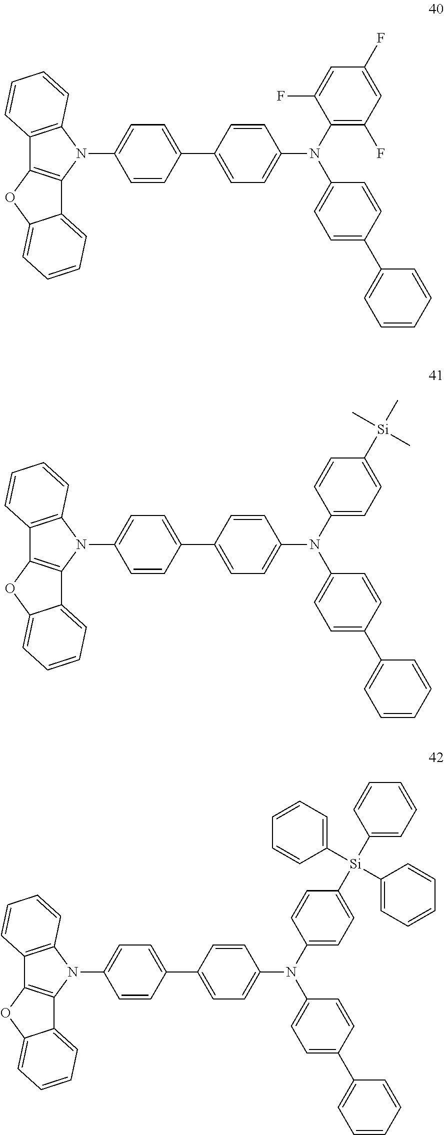 [SMILES: Fc1cc(F)c(N(c2ccc(-c3ccccc3)cc2)c2ccc(-c3ccc(-n4c5ccccc5c5oc6ccccc6c54)cc3)cc2)c(F)c1 ; C[Si](C)(C)c1ccc(N(c2ccc(-c3ccccc3)cc2)c2ccc(-c3ccc(-n4c5ccccc5c5oc6ccccc6c54)cc3)cc2)cc1 ; c1ccc(-c2ccc(N(c3ccc(-c4ccc(-n5c6ccccc6c6oc7ccccc7c65)cc4)cc3)c3ccc([Si](c4ccccc4)(c4ccccc4)c4ccccc4)cc3)cc2)cc1]

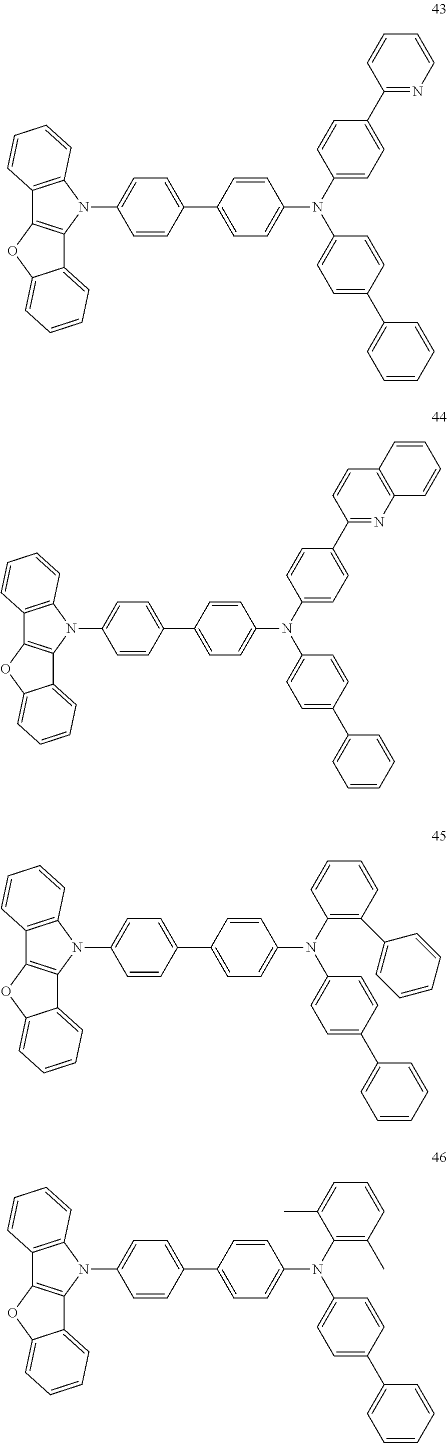 [SMILES: c1ccc(-c2ccc(N(c3ccc(-c4ccc(-n5c6ccccc6c6oc7ccccc7c65)cc4)cc3)c3ccc(-c4ccccn4)cc3)cc2)cc1 ; c1ccc(-c2ccc(N(c3ccc(-c4ccc(-n5c6ccccc6c6oc7ccccc7c65)cc4)cc3)c3ccc(-c4ccc5ccccc5n4)cc3)cc2)cc1 ; c1ccc(-c2ccc(N(c3ccc(-c4ccc(-n5c6ccccc6c6oc7ccccc7c65)cc4)cc3)c3ccccc3-c3ccccc3)cc2)cc1 ; Cc1cccc(C)c1N(c1ccc(-c2ccccc2)cc1)c1ccc(-c2ccc(-n3c4ccccc4c4oc5ccccc5c43)cc2)cc1]

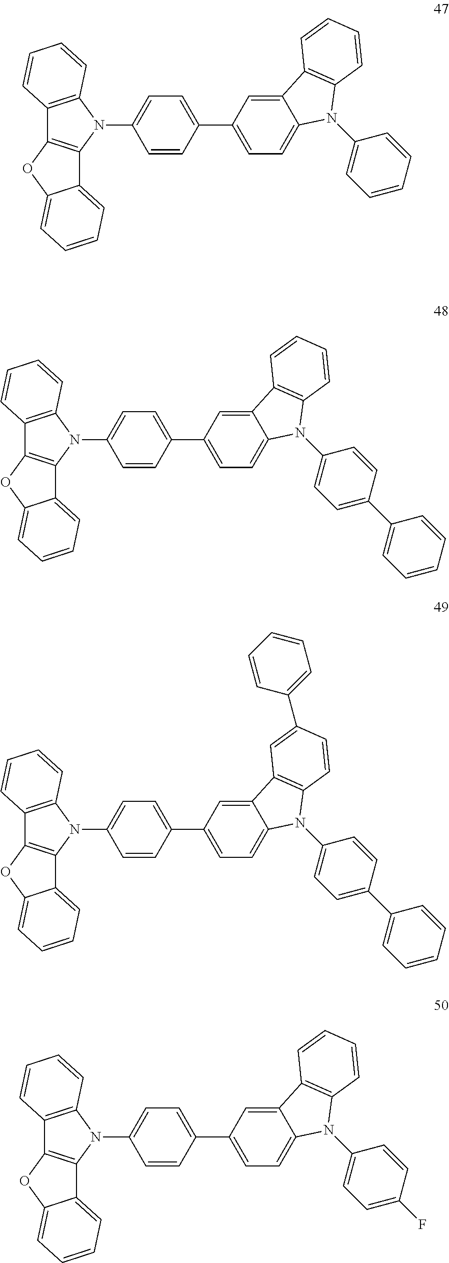 [SMILES: c1ccc(-n2c3ccccc3c3cc(-c4ccc(-n5c6ccccc6c6oc7ccccc7c65)cc4)ccc32)cc1 ; c1ccc(-c2ccc(-n3c4ccccc4c4cc(-c5ccc(-n6c7ccccc7c7oc8ccccc8c76)cc5)ccc43)cc2)cc1 ; c1ccc(-c2ccc(-n3c4ccc(-c5ccccc5)cc4c4cc(-c5ccc(-n6c7ccccc7c7oc8ccccc8c76)cc5)ccc43)cc2)cc1 ; Fc1ccc(-n2c3ccccc3c3cc(-c4ccc(-n5c6ccccc6c6oc7ccccc7c65)cc4)ccc32)cc1]

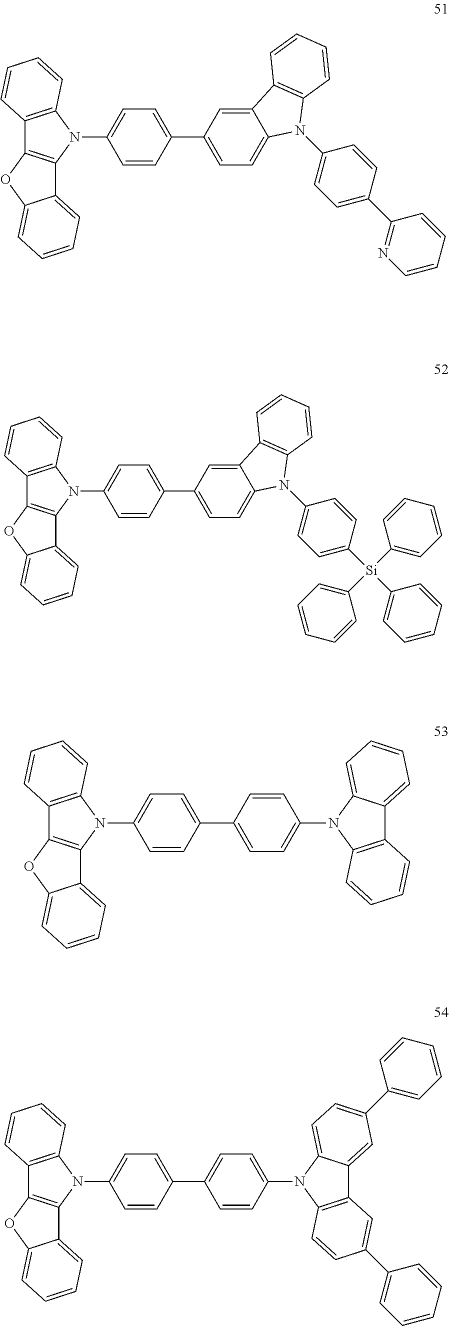 [SMILES: c1ccc(-c2ccc(-n3c4ccccc4c4cc(-c5ccc(-n6c7ccccc7c7oc8ccccc8c76)cc5)ccc43)cc2)nc1 ; c1ccc([Si](c2ccccc2)(c2ccccc2)c2ccc(-n3c4ccccc4c4cc(-c5ccc(-n6c7ccccc7c7oc8ccccc8c76)cc5)ccc43)cc2)cc1 ; c1ccc2c(c1)oc1c3ccccc3n(-c3ccc(-c4ccc(-n5c6ccccc6c6ccccc65)cc4)cc3)c21 ; c1ccc(-c2ccc3c(c2)c2cc(-c4ccccc4)ccc2n3-c2ccc(-c3ccc(-n4c5ccccc5c5oc6ccccc6c54)cc3)cc2)cc1]

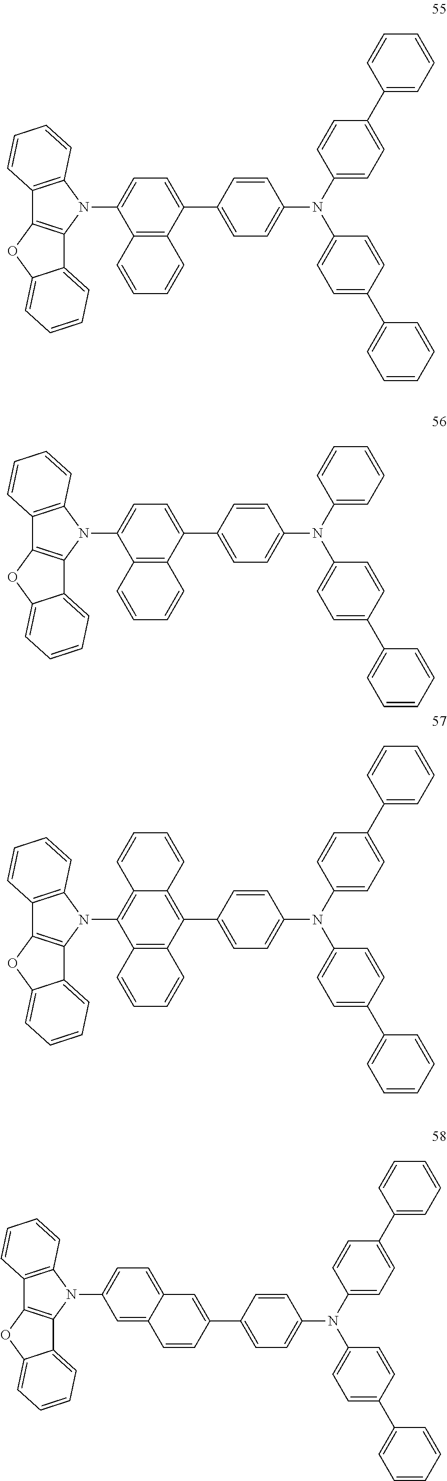

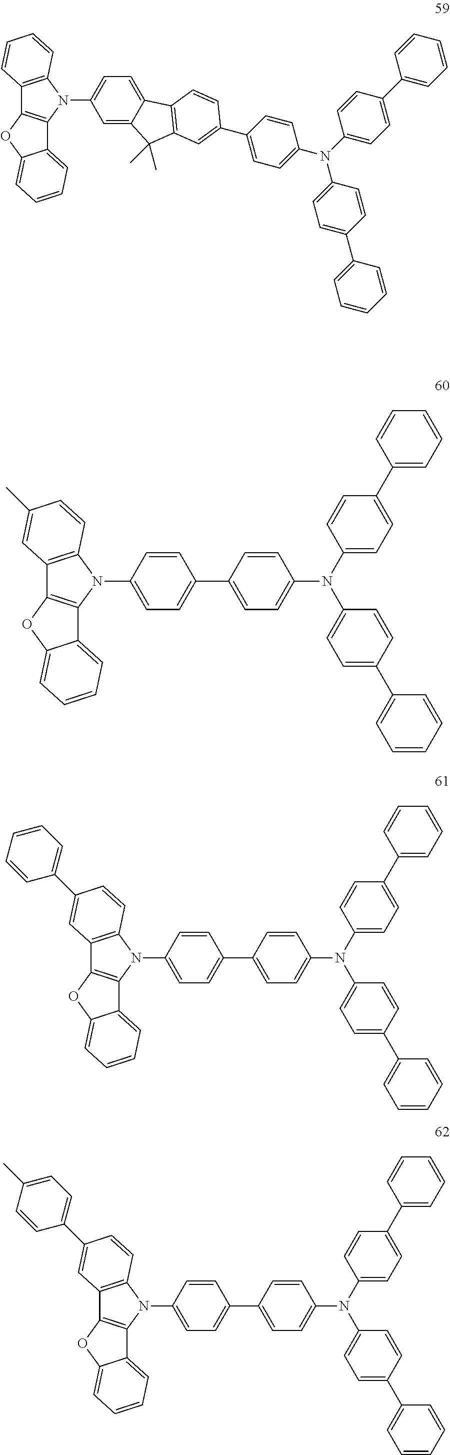 [SMILES: CC1(C)c2cc(-c3ccc(N(c4ccc(-c5ccccc5)cc4)c4ccc(-c5ccccc5)cc4)cc3)ccc2-c2ccc(-n3c4ccccc4c4oc5ccccc5c43)cc21 ; Cc1ccc2c(c1)c1oc3ccccc3c1n2-c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4ccc(-n5c6ccc(-c7ccccc7)cc6c6oc7ccccc7c65)cc4)cc3)cc2)cc1 ; Cc1ccc(-c2ccc3c(c2)c2oc4ccccc4c2n3-c2ccc(-c3ccc(N(c4ccc(-c5ccccc5)cc4)c4ccc(-c5ccccc5)cc4)cc3)cc2)cc1]

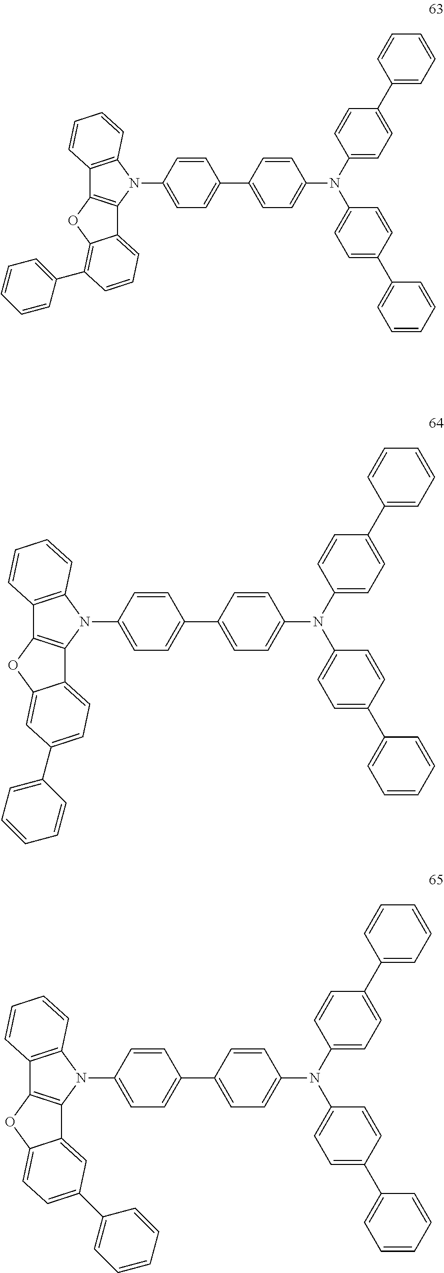 [SMILES: c1ccc(-c2ccc(N(c3ccc(-c4ccccc4)cc3)c3ccc(-c4ccc(-n5c6ccccc6c6oc7c(-c8ccccc8)cccc7c65)cc4)cc3)cc2)cc1 ; c1ccc(-c2ccc(N(c3ccc(-c4ccccc4)cc3)c3ccc(-c4ccc(-n5c6ccccc6c6oc7cc(-c8ccccc8)ccc7c65)cc4)cc3)cc2)cc1 ; c1ccc(-c2ccc(N(c3ccc(-c4ccccc4)cc3)c3ccc(-c4ccc(-n5c6ccccc6c6oc7ccc(-c8ccccc8)cc7c65)cc4)cc3)cc2)cc1]

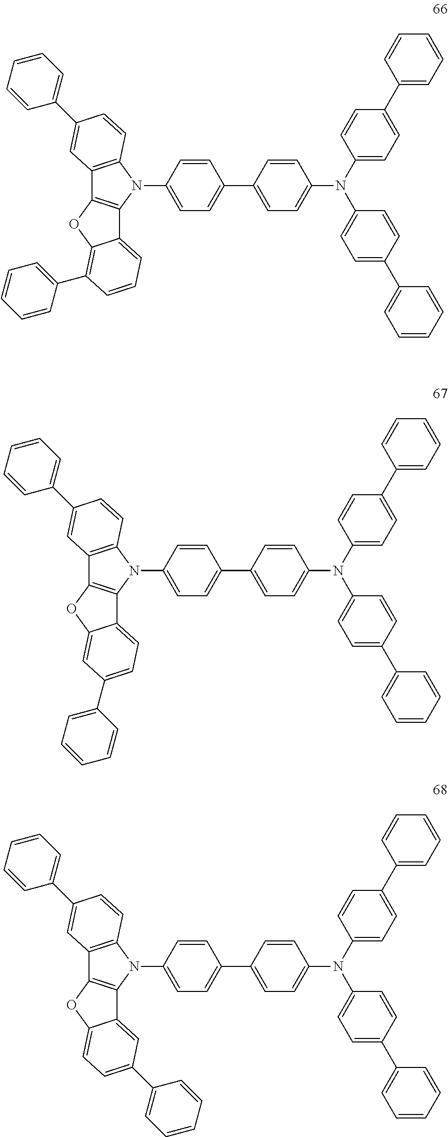 [SMILES: c1ccc(-c2ccc(N(c3ccc(-c4ccccc4)cc3)c3ccc(-c4ccc(-n5c6ccc(-c7ccccc7)cc6c6oc7c(-c8ccccc8)cccc7c65)cc4)cc3)cc2)cc1 ; c1ccc(-c2ccc(N(c3ccc(-c4ccccc4)cc3)c3ccc(-c4ccc(-n5c6ccc(-c7ccccc7)cc6c6oc7cc(-c8ccccc8)ccc7c65)cc4)cc3)cc2)cc1 ; c1ccc(-c2ccc(N(c3ccc(-c4ccccc4)cc3)c3ccc(-c4ccc(-n5c6ccc(-c7ccccc7)cc6c6oc7ccc(-c8ccccc8)cc7c65)cc4)cc3)cc2)cc1]

The compound for an organic electroluminescence device according to an embodiment may be used as a material suitable for the light emitting (e.g., emission) layer of the organic device. In an implementation, the compound for an organic electroluminescence device according to an embodiment may be used as a material in any one layer of stacking layers disposed between the light emitting layer and the anode. Accordingly, a hole transporting property may be improved, and a reduction in driving voltage and high efficiency of the organic electroluminescence device may be embodied.

(Organic Electroluminescence Device)

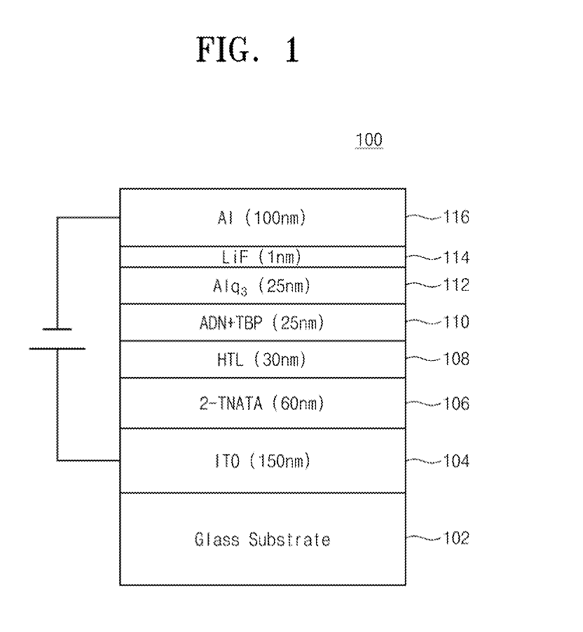

The organic electroluminescence device including the compound for an organic electroluminescence device according to an embodiment will be described. FIG. 1 illustrates a diagram of an organic electroluminescence device according to an embodiment. The organic electroluminescence device 100 may include, e.g., a substrate 102, an anode 104, a hole injection layer 106, a hole transport layer 108, a light emitting layer 110, an electron transport layer 112, an electron injection layer 114, and a cathode 116. In an implementation, the compound for an organic electroluminescence device may be included in the light emitting layer of the organic electroluminescence device. In an implementation, the compound for an organic electroluminescence device may be used or included in any layer of stacking layers disposed between the light emitting layer and the anode.

For example, a case where the compound for an organic electroluminescence device is included in the hole transport layer 108 will be described. The substrate 102, e.g., may be a semiconductor substrate formed of a transparent glass substrate, silicon or the like, and/or a flexible substrate formed of a resin or the like. The anode 104 may be disposed on the substrate 102 and formed by using indium tin oxide (ITO) or indium zinc oxide (IZO). The hole injection layer 106 may be disposed on the anode 104 and may include, e.g., 2-TNATA (4,4′,4 ″-tris[2-naphthyl(phenyl)amino]triphenylamine) or HMTPD (N,N,N′,N′-tetrakis(3-methylphenyl)-3,3′-dimethylbenzidine) or the like. The hole transport layer 108 may be disposed on the hole injection layer 106 and may formed by using the compound for an organic electroluminescence device. The light emitting layer 110 may be disposed on the hole transport layer 108 and may be formed, e.g., by doping TBP (2,5,8,11-tetra-tert-butylperylene) on or in a host material including ADN (9,10-di(2-naphthyl)anthracene) or the like. The electron transport layer 112 may be disposed on the light emitting layer 110 and may be formed of, e.g., a material including Alq3 (tris(8-hydroxyquinolinato)aluminum). The electron injection layer 114 may be disposed on the electron transport layer 112 and may be formed of, e.g., a material including lithium fluoride (LiF). The cathode 116 may be disposed on the electron injection layer 114 and may be formed of metal such as Al or the like, or a transparent material such as indium tin oxide (ITO) or indium zinc oxide (IZO) or the like. The layers may be formed by selecting a suitable film forming method according to material, e.g., vacuum deposition, sputtering, and various coatings.

In the organic electroluminescence device 100 according to an embodiment, the hole transport layer (which may embody a reduction in driving voltage and high efficiency) may be formed by using the compound for an organic electroluminescence device according to an embodiment. In an implementation, the compound for an organic electroluminescence device may be applied even to an organic EL light emitting apparatus of an active matrix using a TFT.

In an implementation, in the organic electroluminescence device 100 according to an embodiment, the compound for an organic electroluminescence device may be used in the light emitting layer, or in any one layer of the stacking layers disposed between the light emitting layer and the anode to embody or achieve a reduction in driving voltage and high efficiency.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

### Example

### Manufacturing Method

### Synthesis of Compound 1

0.45 g of compound A, 1.82 g of compound B, 0.06 g of bis(dibenzylideneacetone)palladium(0) (Pd(dba)₂), 0.09 g of tri-tert-butylphosphine ((t-Bu)₃P), and 0.31 g of sodium tert-butoxide were added to a 100 mL three-neck flask under an argon atmosphere, and heated and refluxed in 45 mL of a toluene solvent for 7 hours. After air cooling, water was added to collect an organic layer, and the solvent was removed by distillation. The obtained crude product was purified by silica gel column chromatography (a mixed solvent of dichloromethane and hexane was used) and then recrystallized with a toluene/hexane mixed solvent to obtain 1.16 g of a white solid of Compound 1 (yield 64%).

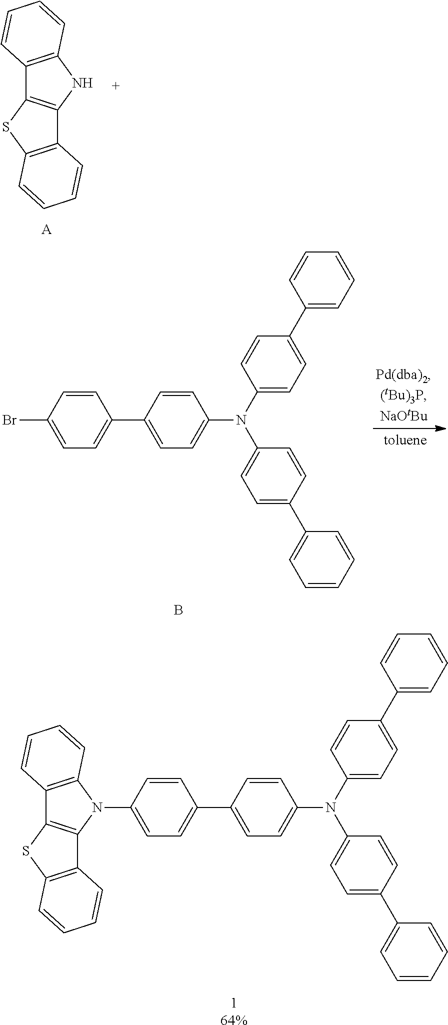

(Identification of Compound 1)

The chemical shift value of Compound 1 measured by ¹H-NMR was 7.91 (d, 1H), 7.87-7.82 (m, 2H), 7.69-7.54 (m, 12H), 7.49-7.38 (m, 5H), and 7.37-7.20 (m, 14H). Further, the molecular weight of Compound 1 measured by FAB-MS was 694.

Synthesis of Compound 35

0.50 g of compound C, 1.33 g of compound B, 0.07 g of bis(dibenzylideneacetone)palladium(0) (Pd(dba)₂), 0.10 g of tri-tert-butylphosphine ((t-Bu)₃P), and 0.35 g of sodium tert-butoxide were added to a 100 mL three-neck flask under an argon atmosphere, and heated and refluxed in 15 mL of a toluene solvent for 7 hours. After cooling in the air, water was added to collect an organic layer, and the solvent was removed by distillation. The obtained crude product was purified by silica gel column chromatography (a mixed solvent of dichloromethane and hexane was used) and then recrystallized with a toluene/hexane mixed solvent to obtain 1.42 g of a white solid of Compound 35 (yield 87%).

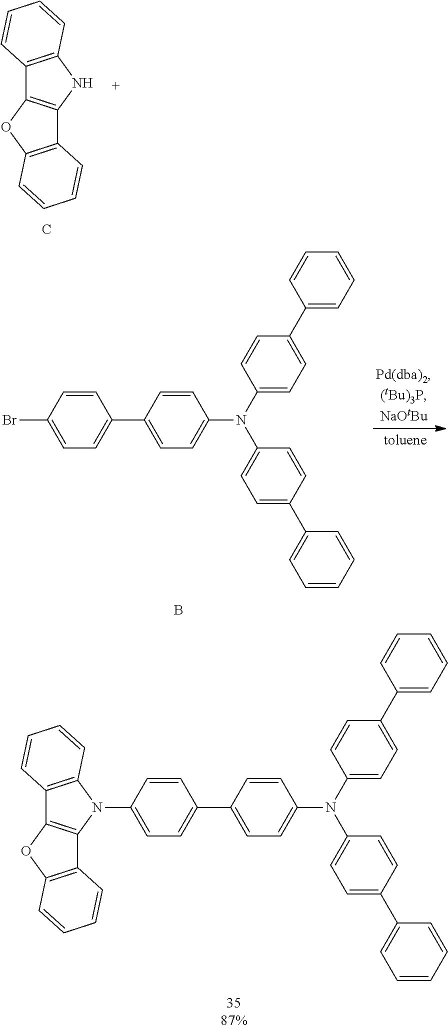

(Identification of Compound 35)

The chemical shift value of Compound 35 measured by ¹H-NMR was 7.88 (d, 1H), 7.85-7.80 (m, 2H), 7.70-7.56 (m, 12H), 7.50-7.38 (m, 5H), and 7.35-7.21 (m, 14H). Further, the molecular weight of Compound 35 measured by FAB-MS was 679.

Synthesis of Compound 13

0.71 g of compound A, 1.26 g of compound D, 0.09 g of bis(dibenzylideneacetone)palladium(0) (Pd(dba)₂), 0.10 g of tri-tert-butylphosphine ((t-Bu)₃P), and 0.46 g of sodium tert-butoxide were added to a 100 mL three-neck flask under an argon atmosphere, and heated and refluxed in 30 mL of a toluene solvent for 7 hours. After cooling in the air, water was added to collect an organic layer, and the solvent was removed by distillation. The obtained crude product was purified by silica gel column chromatography (a mixed solvent of dichloromethane and hexane was used) and then recrystallized with a toluene/hexane mixed solvent to obtain 1.58 g of a white solid of Compound 13 (yield 93%).

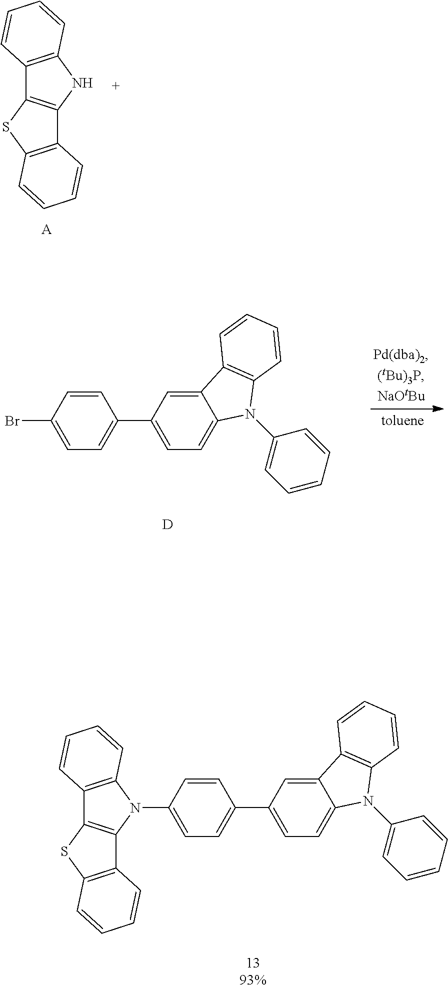

(Identification of Compound 13)

The chemical shift value of Compound 13 measured by ¹H-NMR was 8.32 (d, 1H), 8.20 (d, 1H), 7.93-7.78 (m, 4H), 7.70-7.56 (m, 8H), 7.52-7.36 (m, 5H), and 7.35-7.19 (m, 5H). Further, the molecular weight of Compound 13 measured by FAB-MS was 540.

Organic electroluminescence devices of Examples 1 to 3 were formed by using the Compounds 1, 35, and 13. In addition, compounds C1 and C2, below, were used to form the organic electroluminescence devices of Comparative Examples 1 and 2. Compound C1 has an indolobenzopyrrole moiety. Compound C2 has an indolobenzothiophenyl moiety.

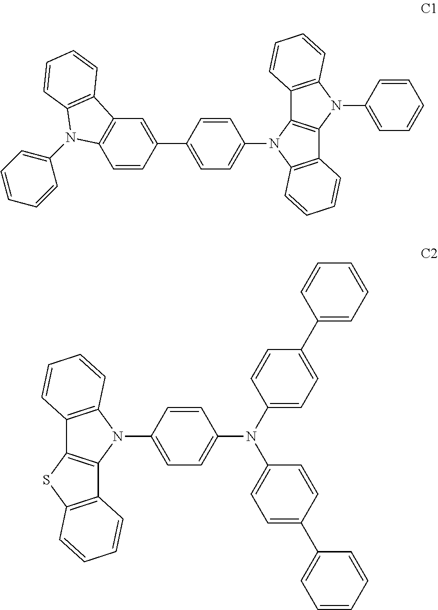 [SMILES: c1ccc(-n2c3ccccc3c3cc(-c4ccc(-n5c6ccccc6c6c5c5ccccc5n6-c5ccccc5)cc4)ccc32)cc1 ; c1ccc(-c2ccc(N(c3ccc(-c4ccccc4)cc3)c3ccc(-n4c5ccccc5c5sc6ccccc6c54)cc3)cc2)cc1]

The organic electroluminescence devices were formed by the aforementioned manufacturing method using Examples 1 to 3 and Comparative Examples 1 and 2 as the hole transport material. In particular, a transparent glass substrate was used as the substrate 102, the anode 104 was formed of ITO having a thickness of 150 nm, the hole injection layer 106 was formed of TNATA having a thickness of 60 nm, the hole transport layer 108 having a thickness of 30 nm was formed with the compounds of Examples 1 to 3 and Comparative Examples 1 and 2, the light emitting layer 110 was formed having a thickness of 25 nm by doping TBP in an amount of 3% in ADN, the electron transport layer 112 was formed of Alq3 having a thickness of 25 nm, the electron injection layer 114 was formed of LiF having a thickness of 1 nm, and the cathode 116 was formed of Al having a thickness of 100 nm.

Voltage and current efficiencies of the manufactured organic electroluminescence devices were evaluated. Further, current efficiency is a value at 10 mA/cm².

TABLE 1

| | Voltage | Light emitting | Half-life |
|---|---|---|---|
| | (V) | efficiency (cd/A) | (hr) |
| Example 1 | 5.9 | 7.1 | 1,400 |
| Example 2 | 6.0 | 7.0 | 1,500 |
| Example 3 | 6.0 | 6.5 | 1,600 |
| Comparative | 6.5 | 6.4 | 1,200 |
| Example 1 | | | |
| Comparative | 6.5 | 6.3 | 1,000 |
| Example 2 | | | |

As may be seen in Table 1, the compounds of Examples 1 to 3 drove the organic electroluminescence device at a low voltage, as compared to the compounds of Comparative Examples 1 and 2. Further, the compounds of Examples 1 to 3 showed high current efficiency, as compared to those of Comparative Examples 1 and 2. Particularly, in Example 3 and Comparative Example 1, the triarylamine group (9-phenylcarbazolyl group) was commonly used, but Example 3 showed a low driving voltage and high light emitting efficiency. As a result, a significant effect may be obtained by including the indolobenzothiophene moiety instead of the indolobenzopyrrole moiety.

Further, in comparing Example 1 and Comparative Example 2, when a phenylene group was included as a divalent connection group (e.g., between the triarylamine moiety and the indolobenzothiophene moiety), the driving voltage was low and light emitting efficiency was high, as compared to the case where the connection group was not introduced. The reason is considered that the hole transporting property was improved by expansion of a conjugation system.

By way of summation and review, when the organic electroluminescence device is applied to a display apparatus, it may be desirable for the organic electroluminescence device to be driven at a low voltage and to have a long life-span. Normalization and stabilization of the hole transport layer may be considered to embody a reduction in driving voltage and high efficiency of the organic electroluminescence device. Various compounds, a carbazole derivative or an aromatic amine-based compound, may be used as a hole transport material in the hole transport layer. The compound according to an embodiment may embody an additional reduction in driving voltage and higher efficiency of the device.

The compound for the organic electroluminescence device according to the embodiment may have an indolobenzofuran moiety or an indolobenzothiophene moiety on a triarylamine moiety to form an organic electroluminescence device driven at a low voltage with high efficiency.

Further, a connection or linking group that may perform conjugation between the indolobenzofuran moiety or the indolobenzothiophene moiety and the triarylamine moiety may be introduced to expand a conjugation system and form the organic electroluminescence device having a long life-span.

The embodiments may provide a material for an organic electroluminescence device that is driven at a low voltage with high efficiency.

The compound for the organic electroluminescence device according to an embodiment may include an indolobenzofuran moiety or an indolobenzothiophene moiety (instead of an indolobenzopyrrole moiety) and may also include a triarylamine moiety. Thus, it is possible to improve the hole transporting property, drive the organic electroluminescence device at a low voltage, and embody high light emitting efficiency.

According to the embodiments, it is possible to provide an organic electroluminescence device material driven at a low voltage with high efficiency.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic electroluminescence device, the compound comprising:
a triarylamine moiety, and
a heterocyclic moiety represented by the following Formula (1): 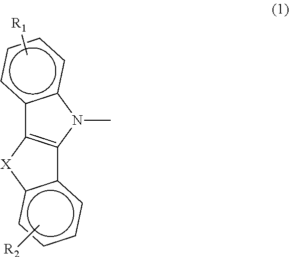
wherein, in Formula (1),
X is an oxygen atom or a sulfur atom, and
R₁ and R₂ are each independently a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom.

2. An organic electroluminescence device, comprising:
an anode;
a cathode;
a light emitting layer between the anode and the cathode; and
at least one stacking layer between the anode and the light emitting layer,
wherein the at least one stacking layer includes the compound for an organic electroluminescence device as claimed in claim 1.

3. An organic electroluminescence device, comprising:
an anode;
a cathode;
a light emitting layer between the anode and the cathode; and
at least one stacking layer between the anode and the light emitting layer,
wherein the light emitting layer includes the compound for an organic electroluminescence device as claimed in claim 1.

4. A compound for an organic electroluminescence device, the compound being represented by the following Formula (2): 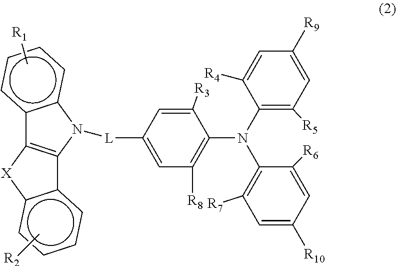
wherein, in Formula (2),
X is an oxygen atom or a sulfur atom,
R₁ and R₂ are each independently a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom,
R₃ to R₁₀ are each independently a hydrogen atom, a substituted or unsubstituted aryl group having 6 to 30 ring carbon atoms, a substituted or unsubstituted heteroaryl group having 3 to 30 ring carbon atoms, a substituted or unsubstituted alkyl group having 1 to 15 carbon atoms, a substituted or unsubstituted silyl group, a cyano group, a halogen atom, or a deuterium atom, and
L is a divalent arylene group.

5. The compound as claimed in claim 4, wherein at least two adjacent ones of R₃ to R₈ are connected to each other to form a saturated or unsaturated ring.

6. An organic electroluminescence device, comprising:
an anode;
a cathode;
a light emitting layer between the anode and the cathode; and
at least one stacking layer between the anode and the light emitting layer,
wherein the at least one stacking layer includes the compound for an organic electroluminescence device as claimed in claim 4.

7. An organic electroluminescence device, comprising:
an anode;
a cathode;
a light emitting layer between the anode and the cathode; and
at least one stacking layer between the anode and the light emitting layer,
wherein the light emitting layer includes the compound for an organic electroluminescence device as claimed in claim 4.
